(19) United States
(12) Patent Application Publication
(10) Pub. No.: US 2021/0035693 A1
(43) Pub. Date: Feb. 4, 2021

(54) METHODS, SYSTEMS, AND APPARATUSES FOR PREDICTING THE RISK OF HOSPITALIZATION

(71) Applicant: McKesson Corporation, San Francisco, CA (US)

(72) Inventors: Naqi Mohammad, Cypress, TX (US); Avinash S. Raju, Houston, TX (US); MD Mahbubur Rahman, Spring, TX (US); William Lopez, Tomball, TX (US); Ramachandran A. Ganesh, Katy, TX (US); Digvijay Yeola, Maharashtra (IN)

(21) Appl. No.: 16/528,087

(22) Filed: Jul. 31, 2019

(51) Int. Cl. G16H 50/30; G16H 10/60; G06N 20/20; G16H 50/20

(52) U.S. Cl. CPC G16H 50/30; G16H 10/60; G16H 50/70; G16H 50/20; G06N 20/20

(57) ABSTRACT

Methods, systems, and apparatuses for improved predictive analytics, such as patient scoring and hospitalization prediction, as described herein. An ensemble classifier may be implemented to predict a hospitalization event for a patient based on healthcare records and demographic information associated with the patient. The ensemble classifier may represent a plurality of machine learning models/classifiers. The prediction generated by the ensemble classifier may be indicative or a range or likelihood that the patient will, or will not, experience a hospitalization event.

### BACKGROUND

Hospitalization is a major cost component creating financial burdens for insurance companies, Medicare, and patients, among other stakeholders in the healthcare industry. In 2015, the U.S. spent an estimated $30.5 billion in total costs for in-patient hospitalization stays for cancer patients. A recent study shows that upwards of 23% of hospitalization events among cancer patients are avoidable. Determining whether a given patient will experience a hospitalization event, especially directly following a surgery or treatment procedure, can be incredibly beneficial for all stakeholders. Thus, what is needed are systems and methods that accurately predict whether a given patient will experience a hospitalization event. These and other considerations are addressed by the present description.

### SUMMARY

It is to be understood that both the following general description and the following detailed description are exemplary and explanatory only and are not restrictive. Methods, systems, and apparatuses for improved predictive analytics, such as patient scoring and hospitalization prediction, are described herein. An ensemble classifier may be implemented to predict a hospitalization event for a patient based on a patient vector representing data extracted from healthcare records and demographic information associated with the patient. The ensemble classifier may represent a plurality of machine learning models/classifiers, such as, for example, a random forest classifier, a naïve Bayes classifier, a gradient boosting machine classifier, an adaptive boosting classifier, or a logistic regression classifier.

The ensemble classifier may be trained using a training dataset including a plurality of patient vectors for a plurality of patients. The prediction generated by the ensemble classifier may be a patient score that is indicative or a range or likelihood that the patient will, or will not, experience a hospitalization event. The patient score may be determined by the ensemble classifier in addition to a meta-classifier implementing a logistic regression algorithm. The patient score may be provided to a reporting subsystem accessible by healthcare providers and practitioners.

This summary is not intended to identify critical or essential features of the present description, but merely to summarize certain features and variations thereof.

Other details and features will be described in the sections that follow.

### BRIEF DESCRIPTION OF THE DRAWINGS

The accompanying drawings, which are incorporated in and constitute a part of the present description serve to explain the principles of the methods and systems described herein:

FIG. 1 shows an example workflow;

FIG. 2 shows an example system;

FIG. 3 shows example data tables;

FIGS. 4A-4C show example diagrams;

FIGS. 5A-5C show example diagrams;

FIG. 6 shows an example bar graph;

FIG. 7 shows an example data table;

FIG. 8 shows an example line graph;

FIG. 9 shows an example system;

FIG. 10 shows an example method;

FIG. 11 shows an example method;

FIG. 12 shows an example method; and

FIG. 13 shows a block diagram of an example computing device.

### DETAILED DESCRIPTION

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another configuration includes from the one particular value and/or to the other particular value. Similarly, when values are expressed as approximations, by use of the antecedent “about,” it will be understood that the particular value forms another configuration. It will be further understood that the endpoints of each of the ranges are significant both in relation to the other endpoint, and independently of the other endpoint.

“Optional” or “optionally” means that the subsequently described event or circumstance may or may not occur, and that the description includes cases where said event or circumstance occurs and cases where it does not.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal configuration. “Such as” is not used in a restrictive sense, but for explanatory purposes.

It is understood that when combinations, subsets, interactions, groups, etc. of components are described that, while specific reference of each various individual and collective combinations and permutations of these may not be explicitly described, each is specifically contemplated and described herein. This applies to all parts of this application including, but not limited to, steps in described methods. Thus, if there are a variety of additional steps that may be performed it is understood that each of these additional steps may be performed with any specific configuration or combination of configurations of the described methods.

As will be appreciated by one skilled in the art, hardware, software, or a combination of software and hardware may be implemented. Furthermore, a computer program product on a computer-readable storage medium (e.g., non-transitory) having processor-executable instructions (e.g., computer software) embodied in the storage medium. Any suitable computer-readable storage medium may be utilized including hard disks, CD-ROMs, optical storage devices, magnetic storage devices, memresistors, Non-Volatile Random Access Memory (NVRAM), flash memory, or a combination thereof.

Throughout this application reference is made to block diagrams and flowcharts. It will be understood that each block of the block diagrams and flowcharts, and combinations of blocks in the block diagrams and flowcharts, respectively, may be implemented by processor-executable instructions. These processor-executable instructions may be loaded onto a general purpose computer, special purpose computer, or other programmable data processing apparatus to produce a machine, such that the processor-executable instructions which execute on the computer or other programmable data processing apparatus create a device for implementing the functions specified in the flowchart block or blocks.

These processor-executable instructions may also be stored in a computer-readable memory that may direct a computer or other programmable data processing apparatus to function in a particular manner, such that the processor-executable instructions stored in the computer-readable memory produce an article of manufacture including processor-executable instructions for implementing the function specified in the flowchart block or blocks. The processor-executable instructions may also be loaded onto a computer or other programmable data processing apparatus to cause a series of operational steps to be performed on the computer or other programmable apparatus to produce a computer-implemented process such that the processor-executable instructions that execute on the computer or other programmable apparatus provide steps for implementing the functions specified in the flowchart block or blocks.

Blocks of the block diagrams and flowcharts support combinations of devices for performing the specified functions, combinations of steps for performing the specified functions and program instruction means for performing the specified functions. It will also be understood that each block of the block diagrams and flowcharts, and combinations of blocks in the block diagrams and flowcharts, may be implemented by special purpose hardware-based computer systems that perform the specified functions or steps, or combinations of special purpose hardware and computer instructions.

The present description relates to methods, systems, and apparatuses for improved predictive analytics, such as patient scoring and hospitalization prediction. Oncology treatment in general can have multiple steps depending on a patient's cancer stage, medical history, age, and other factors. After a chemotherapy or radiation treatment event, or following routine clinic visits, a patient may suffer from adverse events resulting in hospitalization. Hospitalization is a major cost component creating financial burdens for insurance companies, Medicare, the patient, and other healthcare industry stakeholders. In 2015, the Agency for Healthcare Research and Quality estimated $30.5 billion in total cost for in-patient hospitalization stays for cancer patients. A recent study has shown that upwards of 23% of hospitalizations among cancer patients are avoidable.

Described herein, among other things, is a data-driven system to identify patients who may be at risk for hospitalization following a clinic visit. One goal of the present methods and systems is to assist providers with identifying patients at greater risk of hospitalization so that patient care can be managed within the oncology community or outpatient setting as opposed to the hospital. The U.S. Center for Medicare & Medicaid Innovation is developing new payment and delivery models designed to improve the effectiveness and efficiency of specialty care. The Oncology Care Model is one such improvement, which aims to provide higher quality, more highly coordinated oncology care at the same or lower cost to Medicare.

Another goal of the present methods and systems is to reduce the cost of care while improving quality and patient outcomes. Many healthcare practices use Electronic Health Record (EHR) systems to log patients' health-related data in digital repositories. The present methods and systems leverage the ubiquity of EHR data and machine learning models to improve predictive analytics with respect to, for example, patient scoring and hospitalization prediction. Many groups of patients diagnosed with various diseases are at-risk for experiencing an avoidable hospitalization event. For example, oncology patients have a high risk for experiencing an avoidable hospitalization event due to many known/unknown complications during a treatment and/or a procedure. The present methods and systems may be used to predict future medical complications and costly events (e.g. hospitalization) based on previous data and patient experiences. The present methods and systems may thus assist in building awareness, improving patient care, aiding doctors for intervention, and reducing overall costs of treatment. While the present methods and systems utilize complex machine learning models that are dependent on many calculations and sub-systems, a user-friendly front-end reporting tool is provided herein to allow healthcare practices and physicians to quickly and easily access the predictions and related data produced using the present methods and systems. The present methods and systems provide a complete workable solution designed to be deployable and functional on most available technology platforms in place today. The present methods and systems may include a per-patient history-based and a mobile-based reporting system, which allows healthcare professionals to easily retrieve reports using their mobile phones and track patient history over a period of time.

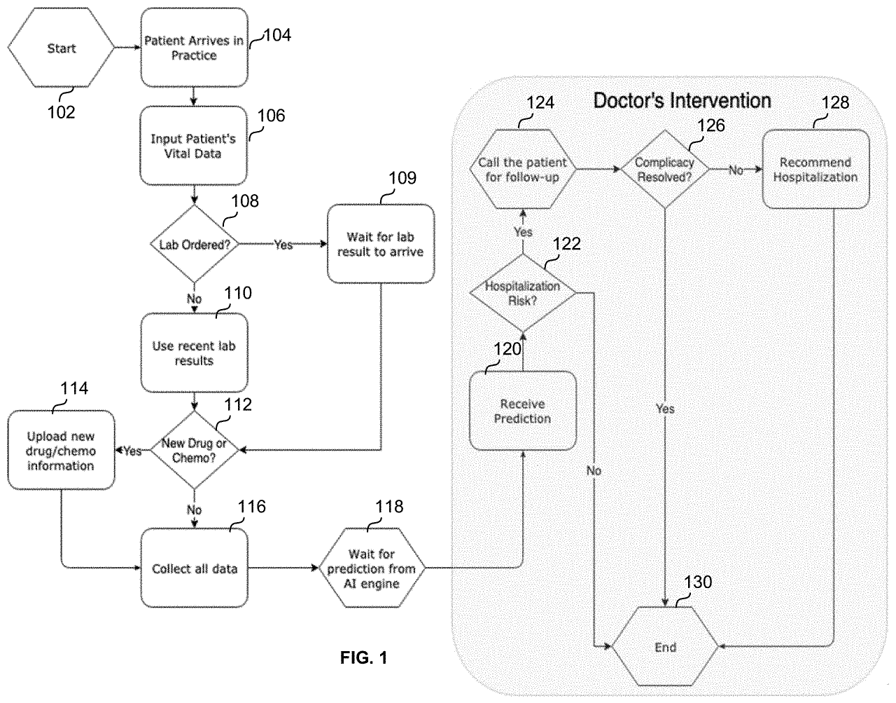

Turning now to FIG. 1, an example workflow 100 for improved predictive analytics, such as patient scoring and hospitalization prediction, is shown. The start 102 of the workflow 100 begins at step 104 with a clinic visit by a patient. Upon each clinic encounter, the healthcare professional takes the patient's vitals and other basic data points at step 106 are collected and stored in the patient medical database (e.g., in a standardized format). At step 118 the collected data is provided (e.g., via the EHR system) to an Artificial Intelligence (AI) engine, which may comprise machine learning, data processing, feature engineering, and/or decision-making subsystems. Once the AI engine creates/generates a prediction on a probability of a hospitalization risk for the patient, at step 120, a report is made available to the corresponding healthcare practice at step 122 (e.g., stored in the EHR system) indicating whether a high probability of hospitalization risk (e.g., greater than 50%) was determined by the AI engine. If a high probability of hospitalization risk is not predicted, then the workflow 100 ends at step 130. Otherwise the workflow 100 proceeds to step 124, where the patient may be contacted for follow-up.

Step 124 may include a clinician intervention stage where a medical care team decides on actions based on the patient's determined probability of hospitalization risk and other clinically relevant data (e.g., the data collected and stored in the patient medical database at step 116). The medical care team may decide to pursue any number of interventions at this point, including but not limited to a review of the current treatment plans and any necessary adjustments that may be needed to avoid adverse events that may result in hospitalization. At step 126 it is determined whether the clinician intervention stage resolved the issue, or issues, that created the hospitalization risk for the patient. If the issue(s) is resolved, then the workflow 100 ends at step 130, otherwise at step 128 the medical care team may recommend hospitalization for the patient.

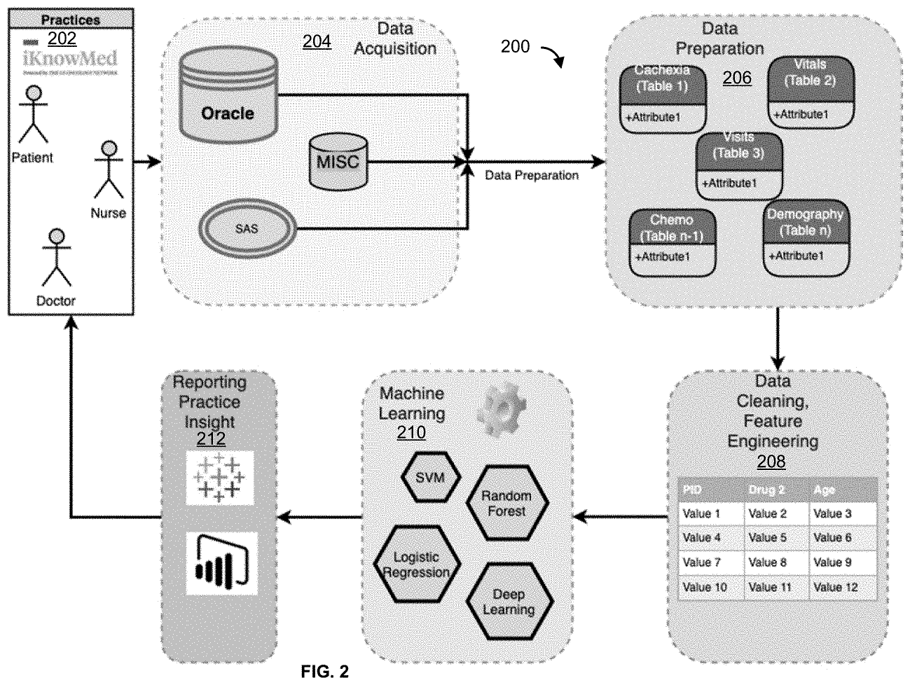

FIG. 2 shows an example system 200 for improved predictive analytics, such as patient scoring and hospitalization prediction. The system 200 may implement the workflow 100 shown in FIG. 1. The system 200 may include an input source 202, representing a variety of medical industry groups and practices and associated patient medical data. The input source 202 may receive data from various sources, such as electronic health records (EHR), claims submission data, Medicare claims, U.S. Census data, and the like (referred to collectively herein as “patient medical data” or “medical data”). The system 200 may include a data acquisition subsystem 204 that may be configured to collect/aggregate patient medical data from the input source 202. The patient medical data may include EHR, which provides most of a patient's medical records; hospital billing data and dates of treatment that may be extracted from claims data; and demographic patient medical data collected/aggregated from various other sources. As an example, the data acquisition subsystem 204 may collect medical data from the various healthcare provider practices that use the iKnowMed® Electronic Health Recording (EHR) system. The data acquisition subsystem 204 may include a transactional database that may be used to store medical data for a plurality of patients, such as vitals, labs, drugs, performance status, pain, disease state, and the like. Patient vitals may be comprised of many different attributes, such as blood pressure, body temperature, pulse rate, heartbeat, weight, height, drug/medications, and the like. Table 1 shows example medical data points that may be acquired from a healthcare practice that uses the iKnowMed® software.

TABLE 1

| Attribute | Data Logged |
|---|---|
| Visit Statistics | Number of time the patient visited the practice, recent visit |
| | dates and treatments performed etc. |
| Patient vitals | Pulse oximetry, blood pressure, pulse, height, weight, pain |
| | score, temperature, and respiratory rate. |
| Labs | Hemoglobin, hematocrit, white blood cells, platelets, mean |
| | corpuscular volume, blood urea nitrogen, creatinine, |
| | sodium, carbon dioxide, ALT, AST, alkaline phosphatase, |
| | bilirubin, calcium, albumin, and GGT. |
| Drug administrations | Non-clinical trial chemo and hormonal drugs |
| | administration, oral drug prescription orders, and other |
| | traditional drugs used in the supportive care setting. |
| Metastatic location | The site location of where a cancer has metastasized, such |
| | as the bone, liver, adrenal gland, or lung. |
| Charlson Comorbidity | An index score is calculated for the patient based on |
| Index | comorbidities present from: Diabetes mellitus, liver disease, |
| | malignancy, AIDS, chronic kidney disease, congestive |
| | heart failure, myocardial infarction, COPD, peripheral |
| | vascular disease, CVA or TIA, dementia, hemiplegia, |
| | connective tissue disease and peptic ulcer disease. |
| | Data points are collected using Total View 2 and iKnowMed |
| | data |

As another example, the data acquisition subsystem 204 may collect medical data from the U.S. Oncology Network, which provides access to records of vital patient medical data, laboratory test data, diagnosis and staging (e.g., ranging from 0 to 4 depending on the severity and spread of the tumor), and the like. When a given patient is diagnosed with an advanced stage cancer (e.g., usually stage IV), the diagnosis indicates progression of disease and likely tumor metastasis. The metastasis to a certain site (e.g., location in the body) can have a strong correlation with the risk of hospitalization for the given patient. Further, certain demographic groups and geographic areas may be at a higher risk for hospitalization (e.g., based on diet, environmental factors, genetics, etc.). To capture the variability among demographic groups and geographic areas, the data acquisition subsystem 204 may collect census data from the U.S. Census database, for example, and add related tags to patient medical data (e.g., EHR) depending on a given patient's location of residence.

As further example, the data acquisition subsystem 204 may collect medical data relating to concomitant illness(es) from a given patient's previous disease history. When an oncology patient is diagnosed with another disease aside from cancer, such as diabetes, cardiac, pulmonary, or hepatic problems, such a concomitant illness may complicate the given patient's overall health condition and lead to a hospitalization event. To capture the impact of concomitant illness(es), the data acquisition subsystem 204 may assign a score to a given patient based on his or her overall health condition. As an example, the data acquisition subsystem 204 may assign a score based on the Charlson Comorbidity Index.

The data acquisition subsystem 204 may also determine whether a given patient was actually admitted to a hospital for the purpose of training the machine learning model(s) described herein. Information indicative of whether a given patient was actually admitted to a hospital can be retrieved/extracted from hospital billing information. For example, the data acquisition subsystem 204 may medical data relating to billing and claims information from the U.S. Centers for Medicare & Medicaid Services (CMS) to identify the patients who were admitted to a hospital in a certain period of time (e.g., a collection of episodes of care).

Raw patient medical data collected/aggregated from the input source 202 by the data acquisition subsystem 204 may require cleaning/preparation in order to make the patient medical data more useful. The system 200 may include a data preparation sub-system 206 that may be configured for initial cleaning of patient medical data and generating intermediate data staging and temporary tables in a database of the data preparation sub-system 206. For example, the data preparation sub-system 206 may divide the patient medical data into multiple subsets of patient medical data and store each subset in a different table in the database. As further examples, the data preparation sub-system 206 may standardize the raw patient medical data (e.g., convert to a common format/structure); determine one or more feature calculations (e.g., determine a patient's BMI from their associated height/weight); perform feature engineering (e.g. determine a duration of disease); classify charted values (e.g. Hemoglobin numeric values are classified as very-low, low, normal, high and very-high); determine an age at diagnosis; determine a residential zip code; a combination thereof, and/or the like.

An example of data cleaning/preparation performed by the data acquisition subsystem 204 may relate to patient medical data for one or more patients diagnosed with breast cancer. Certain breast cancers are classified into two groups: 1) invasive and 2) non-invasive, based on a histology report. The data acquisition subsystem 204 may generate an indication for histology type among one cohort of cancer patients. For example, breast cancer patients with the following types may be considered “invasive” while all other types may be considered “non-invasive”: ‘Invasive ductal carcinoma’, ‘Invasive lobular carcinoma’, ‘Invasive mammary’, ‘Inflammatory carcinoma’, ‘Tubular carcinoma’, ‘Medullary carcinoma’, ‘Metaplastic carcinoma’, ‘Mucinous (colloid) carcinoma’, ‘Papillary carcinoma’, Squamous cell carcinoma', ‘Secretory carcinoma’, ‘Undifferentiated’, ‘Adenoid cystic carcinoma’, ‘Cribriform carcinoma’, ‘Apocrine carcinoma’, ‘Invasive ductal adenocarcinoma’, ‘Invasive ductal adenocarcinoma with lobular; Right breast’, ‘Invasive lobular carcinoma of the right breast’, ‘Invasive ductal carcinoma of the left breast’, ‘Invasive papillary carcinoma’, and ‘Right invasive ductal carcinoma and Left focal usual ductal hyperplasia.’

Another example of data cleaning/preparation performed by the data acquisition subsystem 204 may relate to determining a given patient's performance status. One method of determining performance status may be based on the Eastern Cooperative Oncology Group (ECOG) performance status rating system, which uses a simple measure of functional status of an oncology patient and is commonly used as a prognostic tool, as a selection criterion for cancer research, and to help determine treatment. The ECOG performance status rating system uses scores ranging from 0 to 5, which correlate with scores from the Karnofsky Scale's range of 0-100, as shown in Table 2 below. The score from the Karnofsky Scale may be extracted from the raw patient medical data. As a matter of standardization, the data acquisition subsystem 204 may convert the Karnofsky Scale scores to an ECOG performance status rating using the following conversion table. The data acquisition subsystem 204 may store each patient's determined performance status in the database.

TABLE 2

| Karnofsky Scale | ECOG |
|---|---|
| 90-100 | 0 |
| 70-80 | 1 |
| 50-60 | 2 |
| 30-40 | 3 |
| 10-20 | 4 |

The system 200 may include a data cleaning and feature engineering subsystem 208 that may be configured to prepare medical data for input into the machine learning subsystem 210. For example, the data cleaning and feature engineering subsystem 208 may generate a data point for each of the patients using all corresponding patient medical data. This data point may be referred to as a “vector” of patient medical data that represents all relevant patient medical data for a given patient in a given database table row. Relevant patient medical data may include, for example, vitals, chemotherapy history, metastases, doctor/hospital visits, diagnosis, drugs/medications, concomitant illness(es), hospitalization information, and the like. As another example, the data cleaning and feature engineering subsystem 208 may clean the patient medical data by removing duplicate records for a given patient when multiple entries for the given patient are present in the patient medical data. The data cleaning and feature engineering subsystem 208 may also eliminate any features (e.g., data points within the patient medical data) that are present within the patient medical data less than a threshold amount of times. For example, a feature having 10 or fewer values may not contribute significantly towards a hospitalization prediction. Additionally, the data cleaning and feature engineering subsystem 208 may generate a correlation heat map to visualize relationships among variables/features. The data cleaning and feature engineering subsystem 208 may also check for multi-collinearity between two variables/features and eliminate one of the two by calculating a variance inflation factor (e.g., based on the variable/feature having a higher calculated variance inflation factor).

The data cleaning and feature engineering subsystem 208 may be further configured to perform feature engineering. The machine learning model(s) described herein may function as a binary classifier that predicts either a hospitalization or non-hospitalization event. The machine learning model(s) may use two types of variables: independent variables and dependent variables. A dependent variable may range between 0 and 1, where a value of ‘1’ indicates the patient was hospitalized after a practice visit and a value of ‘0’ indicates otherwise (e.g., patient not hospitalized). Dependent variables may be predicted using machine learning algorithms and the independent variables/features that are engineered by the data cleaning and feature engineering subsystem 208.

The data cleaning and feature engineering subsystem 208 may generate new independent features or modify existing features that can help better predict the target variable (e.g., a hospitalization event). The data cleaning and feature engineering subsystem 208 may eliminate features that do not have significant effect on the target variable. Since the machine learning model(s) is trained on medical data, grouping and categorizing of patients may help the model make better predictions. For example, certain types of cancer that are prevalent in certain racial groups may indicate cohorts that are at higher risk. Accordingly, the data cleaning and feature engineering subsystem 208 may categorize patients into six major race groups: African American; American Indian; Asian; White; Other; No race indicated. The data cleaning and feature engineering subsystem 208 may categorize other variables that most appropriately make sense to a medical professional. Table 3 shows such example categorical variables and their possible values:

TABLE 3

| Category Variable Name | Values |
|---|---|
| Race category | 1. African American |
| | 2. American Indian |
| | 3. Asian |
| | 4. White |
| | 5. Other |
| | 6. No race indicated |
| Age category | 1. Less than 65 years |
| | 2. 65-74 years |
| | 3. 75-85 years |
| | 4. More than 85 years |
| Duration of disease | 1. New disease (Less than 6 months since diagnosis) |
| | 2. Medium duration disease (Between 6 months and 2 |
| | years since diagnosis) |
| | 3. Old disease (More than 2 years since diagnosis) |
| Zip Code Category | 1. Rural (Zip codes with population less than 2,500) |
| | 2. Urbanless (Zip codes with population between 2,500 |
| | and 20,000) |
| | 3. Urban (Zip codes with population between 20,000 and |
| | 250,000) |
| | 4. Metropolis (Zip codes with population between |
| | 250,000 and 1 million) |
| | 5. Big metropolis (Zip codes with population greater |
| | than 1 million) |
| Cachexia category | 1. No Cachexia |
| | 2. Pre-Cachexia |
| | 3. Cachexia |
| | 4. Refractory Cachexia |
| Drug route category | 1. Intravenous |
| | 2. Oral |
| | 3. Intramuscular |
| | 4. Subcutaneous |
| Systolic blood pressure | 1. Normal (Less than 130 mmHg) |
| | 2. Stage 1 (130-139 mmHg) |
| | 3. Stage 2 (Greater than 140 mmHg) |
| Diastolic blood pressure | 1. Normal (Less than 80 mmHg) |
| | 2. Stage 1 (80-89 mmHg) |
| | 3. Stage 2 (Greater than 90 mmHg) |
| Body mass index | 1. Underweight (Less than 18.5) |
| | 2. Normal weight (18.5-24.9) |
| | 3. Overweight (25-29.9) |
| | 4. Obese (Greater than or equal to 30) |
| Pain category | 1. No pain (pain scale is 0) |
| | 2. Mild pain (pain scale 1-3) |
| | 3. Moderate pain (pain scale 4-6) |
| | 4. Severe pain (pain scale 7-9) |
| | 5. Worst pain (pain scale greater than 10) |
| Respiratory rate category | 1. Low (Less than 13 breaths per minute) |
| | 2. Normal (Between 13 and 25 breaths per minute) |
| | 3. High (Greater than 25 breaths per minute) |
| Heart rate category | 1. Low (less than 60 beats per minute) |
| | 2. Normal (Between 60-83 beats per minute) |
| | 3. High (Greater than or equal to 84 beats per minute) |
| Body temperature category | 1. Low (less than 95° F.) |
| | 2. Normal (between 95° F. and 100.3° F.) |
| | 3. High (greater than 100.4° F.) |
| Drug class category (Breast | 1. Fluoropyrimidine |
| cancer therapy) | 2. Gemcitabine |
| | 3. Platinum |
| | 4. Taxane |
| | 5. Other category |
| Chemotherapy drug counts | 1. One |
| for each patient | 2. Two |
| | 3. Three or more |
| First chemotherapy | 1. Chemotherapy received on office visit (event date) |
| reception date category | 2. Chemotherapy received within 90 days before event |
| | date |
| | 3. Chemotherapy received between 90 to 180 days |
| | before event date |
| | 4. Chemotherapy received between 180 to 270 days |
| | before event date |
| | 5. Chemotherapy received between 270 to 360 days |
| | before event date |
| | 6. Chemotherapy received between 360 to 450 days |
| | before event date |
| | 7. Chemotherapy received greater than 450 days before |
| | event date |
| Last chemotherapy | 1. Chemotherapy received on office visit (event date) |
| reception date category | 2. Chemotherapy received within 7 days before event |
| | date |
| | 3. Chemotherapy received between 7 to 14 days before |
| | event date |
| | 4. Chemotherapy received between 14 to 21 days before |
| | event date |
| | 5. Chemotherapy received between 21 to 28 days before |
| | event date |
| | 6. Chemotherapy received between 28 to 35 days before |
| | event date |
| | 7. Chemotherapy received between 35 to 42 days before |
| | event date |
| | 8. Chemotherapy received between 42 to 49 days before |
| | event date |
| | 9. Chemotherapy received greater than 49 days before |
| | event date |
| Number of encounters for | 1. Zero encounters |
| each patient | 2. 1-3 encounters |
| | 3. 4-6 encounters |
| | 4. 7-9 encounters |
| | 5. 10 or more encounters |
| Charlson Comorbidity | 1. CCI between 0 and 3 |
| Index (CCI) | 2. CCI between 4 and 8 |
| | 3. CCI of 9 |
| | 4. CCI of 10 or more |

Many of the variables shown in Table 3 are time-bounded and have their own expiration period. These variables may constantly change throughout time; therefore, the data cleaning and feature engineering subsystem 208 may only assess vitals recorded within the last 7 days prior to an event date (e.g., a clinic visit) in patient medical data of a given patient record. Similarly, the data cleaning and feature engineering subsystem 208 may review labs data within the last 7 days in the patient medical data, drug treatment within the last 60 days in the patient medical data, Charlson Comorbidity Index for the last 730 days in the patient medical data, cachexia (weight loss) for the last 182 days prior to an event date (clinic visit) in the patient medical data, and/or the like.

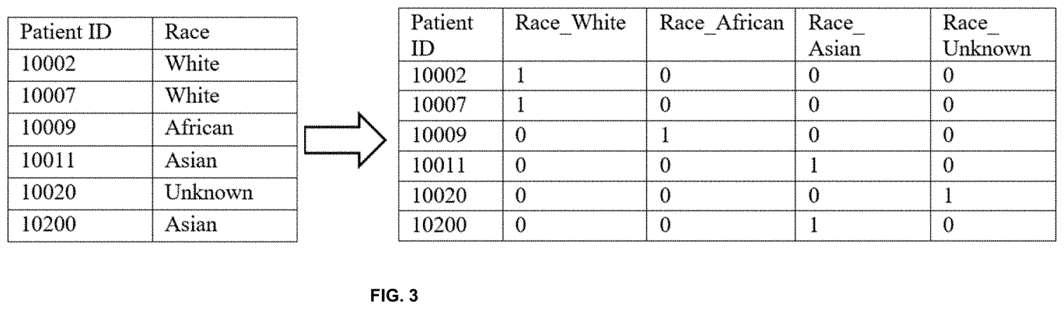

The data cleaning and feature engineering subsystem 208 may convert various categorical variables having text values into numerical format before providing the patient medical data to the machine learning subsystem 210. The data cleaning and feature engineering subsystem 208 may accomplish such data conversion using a process referred to as “one hot encoding,” which generates dummy features for each of the distinct text values in a categorical feature, as shown in FIG. 3. After data conversion, the values of the features may be filled with binary numbers (e.g., dichotomization). As shown in FIG. 3, one variable may be converted to a feature that receives a value of ‘1,’ (e.g., ‘true’) while all other will variables may be converted to respective features that each receive a value of ‘0’ (e.g., ‘false’) in a particular row after conversion. For example, FIG. 3 indicates that all patients are associated with a feature “Race” that has a value of either “White,” “African,” “Asian,” or “Unknown.” Patient ID 1002 indicates a Race of “White.” After conversion, a total of four features are associated with Patient ID 1002: “Race_White,” “Race_African,” “Race_Asian,” and “Race_Unknown.” The feature “Race_White” indicates a value of ‘1’ (e.g., ‘true’), while the remaining features indicate a value of ‘0’ (e.g., ‘false’).

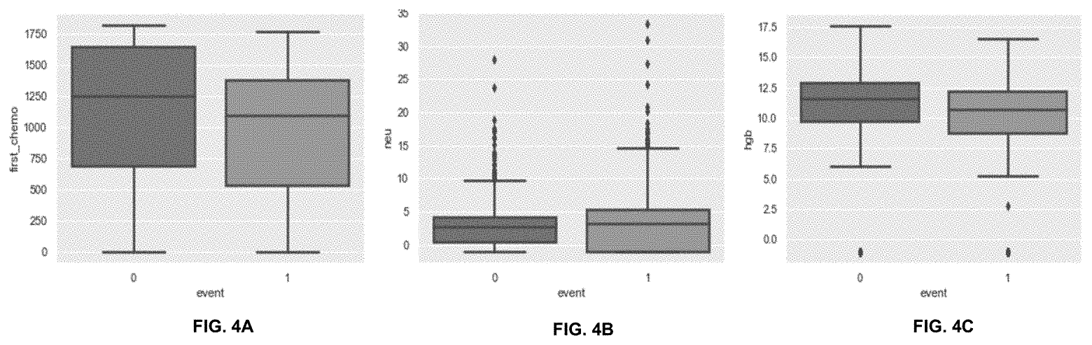

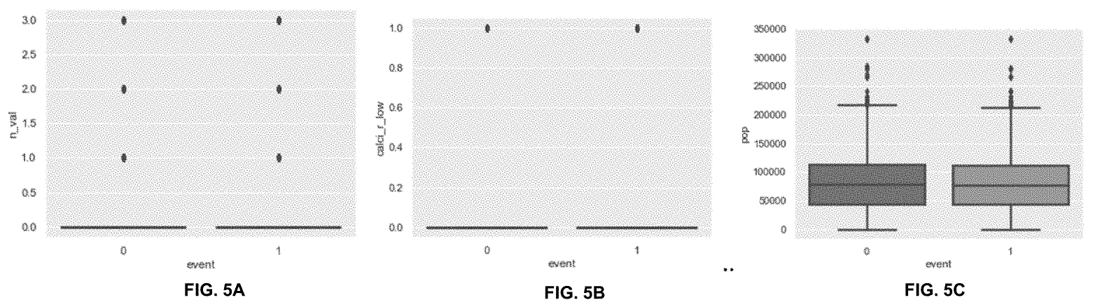

The data cleaning and feature engineering subsystem 208 may use variable elimination techniques to eliminate unimportant variables so they do not add noise to the machine learning model(s). As noted herein, the machine learning model(s) may utilize a binary classifier. In order to determine which features are candidates for elimination, the data cleaning and feature engineering subsystem 208 may generate boxplots for every variable. Example boxplots are shown in FIGS. 4A-4C. A boxplot may contain a mean value, a maximum value, a minimum value, a 75-percentile, a 25-percentile, outlier values, and/or the like. Boxplots may assist in identifying the variations of values among multiple classes. As shown in FIGS. 4A-4C two classes are considered: patients who did not experience a hospitalization event—labeled as class ‘0’ in each boxplot; and patients who did experience a hospitalization event—labeled as class ‘1’ in each boxplot. When boxplots between two classes remain the same for a given variable, the data cleaning and feature engineering subsystem 208 may eliminate that corresponding variable as it may not contribute in separating the classes during training. For example, the boxplots shown in FIGS. 4A-4C indicate that the variables of date since first chemotherapy, neutrophil, and hemoglobin, respectively, have significant impact on probability of a hospitalization event, since the boxplots for the two classes in each of FIGS. 4A-4C are not the same size. In contrast, the boxplots shown in FIGS. 5A-5C indicate that the variables of n-value of tumor, calcium level, and population in neighborhood, respectively, are insignificant in terms of assisting the machine learning model(s) in predicting a probability of a hospitalization event since the boxplots for the two classes in each of FIGS. 5A-5C are the same size.

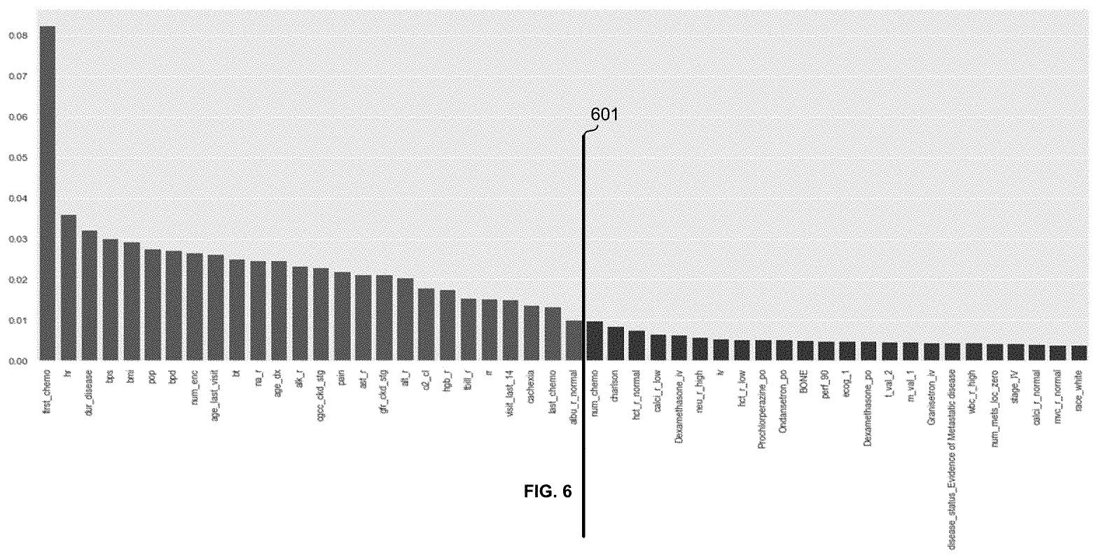

The data cleaning and feature engineering subsystem 208 may generate a feature importance bar chart according to the features' importance rank as shown FIG. 6. As shown in FIG. 6, the features to the left of the line 601 may assist the machine learning model(s) in predicting a probability of a hospitalization event. As also shown in FIG. 6, the most important features that may be determinative with respect to hospitalization or no-hospitalization may be the features furthest to the left of the line 601, such as days since first chemotherapy, heart rate (hr), duration of disease, body mass index (BMI), systolic blood pressure (bps), and/or the like.

Returning to FIG. 2, the machine learning subsystem 210 may be configured to train the machine learning model(s) that may be used to predict a given patient's likelihood of a future hospitalization event. The machine learning subsystem 210 may receive the patient medical data as an input that is used to train the machine learning model(s). The machine learning subsystem 210 may evaluate several machine learning algorithms using various statistical techniques such as, for example, accuracy, precision, recall, Fl-score, confusion matrix, ROC curve, and/or the like. The machine learning subsystem 210 may also perform hyper-parameter tuning to achieve best fitting of the machine learning model(s).

The machine learning model(s) trained and implemented by the machine learning subsystem 210 may include trained Random Forest, Gradient Boosting, Adaptive Boosting, K-Nearest Neighbors, Naïve Bayes, Logistic Regressor Classifier, a combination thereof and/or the like. Gradient Boosting may add predictors to an ensemble (e.g., a combination of two or more machine learning models/classifiers) in sequence to correct each preceding prediction (e.g., by determining residual errors). The K-Nearest Neighbors algorithm may receive each data point and looks at the “k” closest data points. The AdaBoost Classifier may attempt to correct a preceding classifier's predictions by adjusting associated weights at each iteration. The Support Vector Machine—algorithm plots data points in n-dimensional space and identifies a best hyperplane that separates a dataset into two groups (e.g., hospitalized vs. not hospitalized). Logistic Regression may be used to identify an equation that may estimate a probability of hospitalization as a function of the features (e.g., a vector). Gaussian Naïve Bayes draws a decision boundary between two classes based on Bayesian conditional probability theorem. A Random Forest Classifier may consists of a collection of decision trees that are generated randomly using random data sampling and random branch splitting (e.g., in every tree in the forest), and a voting mechanism and/or averaging of outputs from each of the trees may be used to decide about a class.

The machine learning subsystem 210 may use random search and grid search approaches to estimate a best parameter for the machine learning model(s) without overfitting them. For example, in tree-based methods the machine learning subsystem 210 may determine a number of trees, depth of the trees, maximum leaf nodes and so on. The machine learning subsystem 210 may start with a range of values for each of the parameters and use a random search to explore and narrow down a search space by evaluating random subsets of the parameters. Once the search space is minimized, the machine learning subsystem 210 may use a grid search to evaluate every possible combination of parameters in that space.

The machine learning subsystem 210 may select one or more of the machine learning models to generate an ensemble classifier (e.g., an ensemble of one or more classifiers). Selection of the one or more of the machine learning models may be based on each respective models' F-1 score, precision, recall, accuracy, and/or confusion metrics (e.g., minimal false positives/negatives). For example, the ensemble classifier may use Random Forest, Gradient Boosting Machine, Adaptive Boosting, Logistic Regression, and Naïve Bayes models. The machine learning subsystem 210 may use a logistic regression algorithm as a meta-classifier. The meta-classifier may use respective predictions of each model of the ensemble classifier as its features to make a separate prediction of a hospitalization event for a given patient.

The machine learning subsystem 210 may train the ensemble classifier based on the received patient medical data. For example, the machine learning subsystem 210 may train the ensemble classifier to predict results for each of the multiple combinations of variables within the patient medical data. The predicted results may include soft predictions, such as one or more predicted results, and a corresponding likelihood of each being correct. For example, a soft prediction may include a value between 0 and 1 that indicates a likelihood of a hospitalization event, with a value of 1 being a prediction with 100% accuracy that the patient will be hospitalized, and a 0.5 corresponding to a 50% likelihood that the patient will be hospitalized. The machine learning subsystem 210 may make the predictions based on applying the features engineered by the data cleaning and feature engineering subsystem 208 to each of the multiple combinations of variables within the patient medical data.

The meta-classifier may be trained using the predicted results from the ensemble classifier along with the corresponding combinations of variables within the patient medical data. For example, the meta-classifier may be provided with each set of the variables and the corresponding prediction from the ensemble classifier. The meta-classifier may be trained using the prediction from each classifier that is part of the ensemble classifier along with the corresponding combinations of variables.

The meta-classifier may be trained to output improved predictions that are based on the resulting predictions of each classifier of the ensemble classifier based on the same variables. The meta-classifier may then receive a new set of variables/patient medical data and may predict a hospitalization event (i.e., a soft prediction) based on the new set of variables/patient medical data. The prediction by the meta-classifier that is based on the ensemble classifier may include one or more predicted results along with a likelihood of accuracy of each prediction.

The system 200 may include a reporting subsystem 212. Predictions provided by the ensemble classifier and/or the meta-classifier may be provided by the machine learning subsystem 210 to the reporting subsystem 212. The reporting subsystem 212 may generate understandable and human-readable reports so that a healthcare professional may be provided with both concise and detailed insight about a patient's condition. The reporting subsystem 212 may also provide a historical report and trigger points for a patient's results so that the healthcare professional can easily determine a source of a patient's problem that may result in hospitalization.

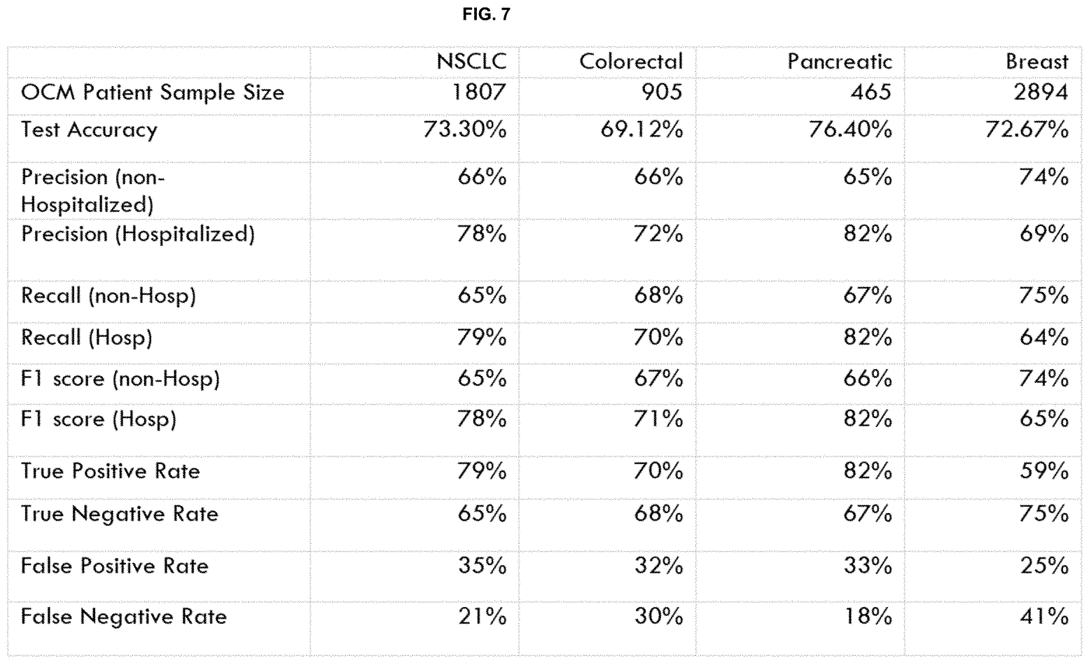

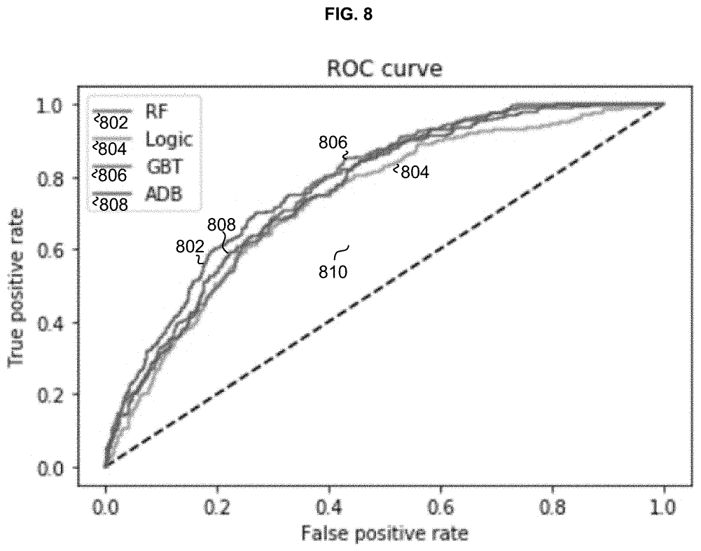

The table shown in FIG. 7 includes findings on different models' performances for different types of diseases, including Breast Cancer, Non-Small-Cell Lung Cancer (NSCLC), Pancreatic Cancer, and Colorectal Cancer patients. The accuracies using the machine learning model(s) described herein were all above 70% for all models. FIG. 8 shows a plot of a receiving operatic characteristic (ROC) curve to visualize the accuracy of the machine learning model(s) described herein, such as the Random Forest model 802, the Logistical Regression model 804, the Gradient Boosted Tree model 806, and the Adaptive Boosting model 808. As FIG. 8 shows, most of the models have an AUC (area under curve) value 810 of 0.7, especially the Gradient Boosted Tree model 806and the Adaptive Boosting model 808.

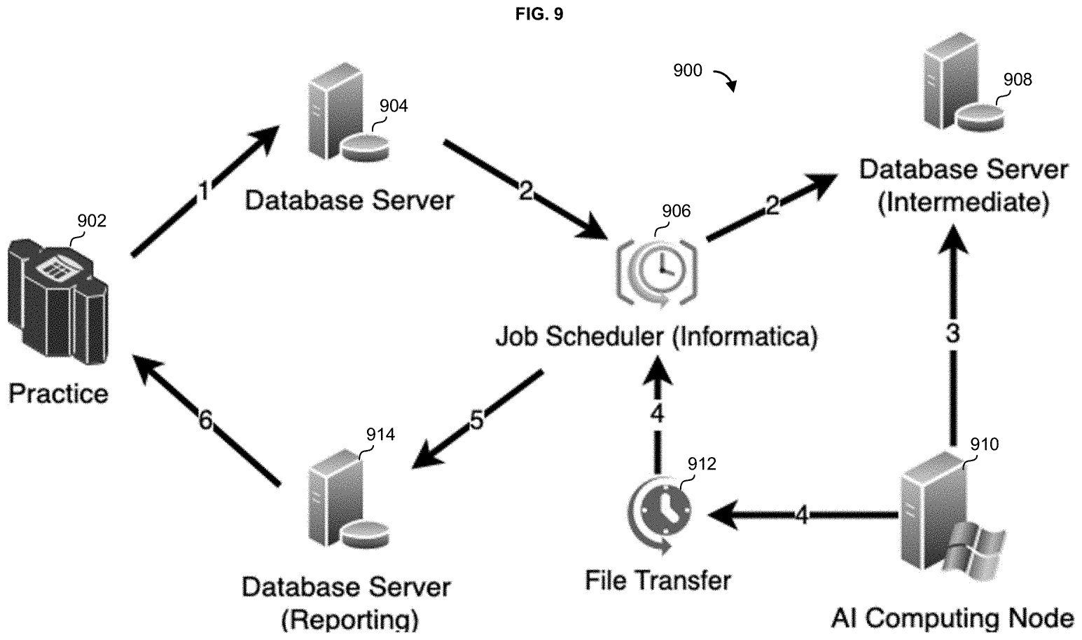

FIG. 9 shows an example system 900 for improved predictive analytics, such as patient scoring and hospitalization prediction, in accordance with the present description. The system 900 may provide a generic system framework that is designed to work best with most of medical facilities and practices 902. Patient medical data may be collected/aggregated from the practices 902 and stored in a database server 904, such as a transactional database. A job scheduler 906 may be used to modify and clean the patient medical data using, for example, SQL scripts, functions and procedures that run on a scheduled basis. The job scheduler 906 may use an Informatica™ database job scheduler that loads cleaner data into an intermediate database 908. The timing of the scheduled jobs is coordinated by the job scheduler 906 in such a way as to not affect the performance of a corresponding live production system during peak utilization hours.

The patient medical data stored in the intermediate database 908 may be provided to an Artificial Intelligence (AI) node 910. The AI node 910 may be a high-performance node with sufficient hardware resources and IT support to perform heavy computing jobs (e.g., during training of a machine learning model) related to machine learning and data processing. The AI node 910 may implement several machine learning models and a meta-classifier to generate output files (e.g., csv or JSON format) and store them in a shared location for the next system to retrieve. The AI node 910 may generate output files for each cancer type (i.e. one for breast cancer, one for colorectal cancer, and so on) indicated in the patient medical data.

The output files may be provided by the AI node 910 using a file transfer protocol 912, such as a secure file transfer system during off-hours, and loaded into the job scheduler 906. The job scheduler 906 may process the output files and subsequently load results of the processing into a reporting database server 914. There may be two repositories in the reporting database server 914; one repository may store only new data (e.g., the reporting database server 914 wipes old data and loads new data) and the other repository may store both new and old data (e.g., for historical tracking purposes). The reporting database server 914 may then provide the results of the processing to practices 902.

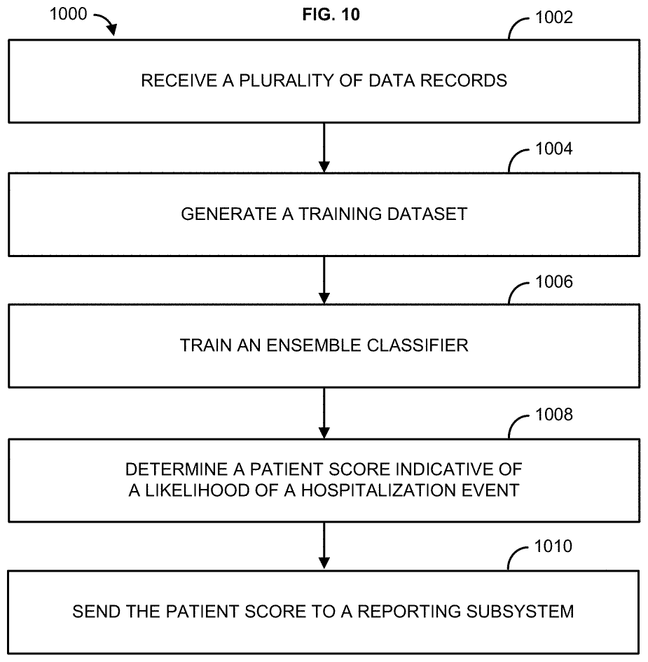

FIG. 10 is a flowchart of a method 1000 for improved predictive analytics, such as patient scoring and hospitalization prediction, in accordance with the present description. Method 1000 may be implemented using the workflow 100 of FIG. 1, the system 200 of FIG. 2, and/or the system 300 of FIG. 9. At step 1002, a plurality of data records associated with a plurality of patients may be received by a computing device. The plurality of data records may include EHR data, demographic data, and the like. At step 1004, a training dataset may be generated. The training dataset may be based on the plurality of data records. The training dataset may include a plurality of vectors each corresponding to a respective patient of the plurality of patients. Each of the plurality of vectors may be indicative of a health condition score based on, for example, the Charlson Comorbidity Index. Each of the plurality of vectors may be indicative of a Karnofsky Scale score. A performance status score ranging from 0 to 4 may be determined for each of the plurality of vectors based on the Karnofsky Scale score for each of the plurality of vectors.

At step 1006, an ensemble classifier may be trained. The ensemble classifier may be trained using the training dataset. The ensemble classifier may be representative of one or more classifiers such as, for example, a random forest classifier, a naïve Bayes classifier, a gradient boosting machine classifier, an adaptive boosting classifier, or a logistic regression classifier. The one or more classifiers may be selected for the ensemble based on one or more of an F-1 score, a precision, a recall, an accuracy, or a confusion metric for each of the one or more classifiers.

At step 1008, a patient score indicative of a likelihood of a hospitalization event for a subject patient may be determined. The patient score may be based on the trained ensemble classifier being applied to a patient vector for the subject patient. The patient score may be determined by generating one or more dependent patient scores, each indicative of a respective likelihood of the hospitalization event for the subject patient. The one or more dependent patient scores may be based on the one or more classifiers applied to the subject vector for the subject patient. The patient score may be determined based on a meta-classifier applied to the one or more dependent patient scores. The meta-classifier may include a logistic regression algorithm. At step 1010, the patient score may be provided to a second computing device, such as a reporting subsystem.

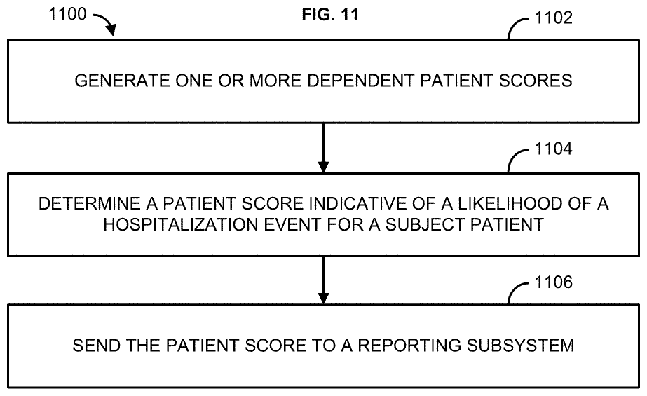

FIG. 11 is a flowchart of a method 1100 for improved predictive analytics, such as patient scoring and hospitalization prediction, in accordance with the present description. Method 1100 may be implemented using the workflow 100 of FIG. 1, the system 200 of FIG. 2, and/or the system 300 of FIG. 9. At step 1102, one or more dependent patient scores each indicative of a respective likelihood of a hospitalization event for a subject patient may be determined. The one or more dependent patient scores may be based on a trained ensemble classifier being applied to a patient vector for the subject patient. The one or more dependent patient scores may each be indicative of a respective likelihood of the hospitalization event for the subject patient. The one or more dependent patient scores may be based on one or more classifiers applied to the subject vector for the subject patient. At step 1104, a patient score indicative of the likelihood of the hospitalization event for the subject patient may be determined. The patient score may be determined based on a meta-classifier applied to the one or more dependent patient scores. The meta-classifier may include a logistic regression algorithm.

A plurality of data records associated with a plurality of patients may be received by the computing device, and a training dataset may be generated. The training dataset may be based on the plurality of data records. The training dataset may include a plurality of vectors each corresponding to a respective patient of the plurality of patients. Each of the plurality of vectors may be indicative of a health condition score based on, for example, the Charlson Comorbidity Index. Each of the plurality of vectors may be indicative of a Karnofsky Scale score. A performance status score ranging from 0 to 4 may be determined for each of the plurality of vectors based on the Karnofsky Scale score for each of the plurality of vectors.

An ensemble classifier may be trained. The ensemble classifier may be trained using the training dataset. The ensemble classifier may be representative of one or more classifiers such as, for example, a random forest classifier, a naïve Bayes classifier, a gradient boosting machine classifier, an adaptive boosting classifier, or a logistic regression classifier. The one or more classifiers may be selected for the ensemble based on one or more of an F-1 score, a precision, a recall, an accuracy, or a confusion metric for each of the one or more classifiers. At step 1106, the patient score may be provided to a second computing device, such as a reporting subsystem.

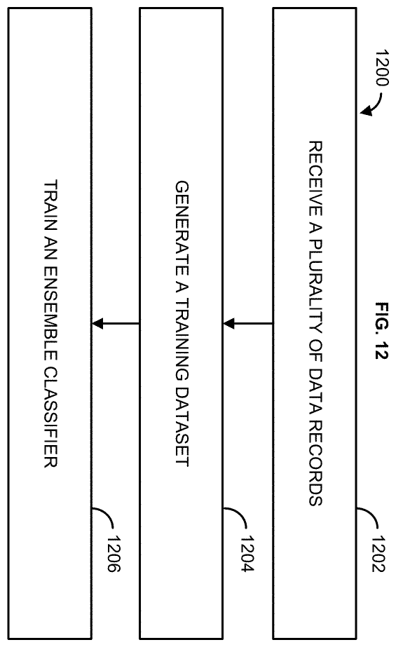

FIG. 12 is a flowchart of a method 1200 for improved predictive analytics, such as patient scoring and hospitalization prediction, in accordance with the present description. Method 1200 may be implemented using the workflow 100 of FIG. 1, the system 200 of FIG. 2, and/or the system 300 of FIG. 9. At step 1202, a plurality of data records associated with a plurality of patients may be received by a computing device. The plurality of data records may include EHR data, demographic data, and the like. At step 1204, a training dataset may be generated. The training dataset may be based on the plurality of data records. The training dataset may include a plurality of vectors, each including a standardized Karnofsky Scale score and a health condition score corresponding to a respective patient of the plurality of patients. Each of the plurality of vectors may be indicative of a health condition score based on, for example, the Charlson Comorbidity Index. A performance status score ranging from 0 to 4 may be determined for each of the plurality of vectors based on the Karnofsky Scale score for each of the plurality of vectors.

At step 1206, an ensemble classifier may be trained. The ensemble classifier may be trained using the training dataset. The ensemble classifier may be representative of one or more classifiers such as, for example, a random forest classifier, a naïve Bayes classifier, a gradient boosting machine classifier, an adaptive boosting classifier, or a logistic regression classifier. The one or more classifiers may be selected for the ensemble based on one or more of an F-1 score, a precision, a recall, an accuracy, or a confusion metric for each of the one or more classifiers.

A patient score indicative of a likelihood of a hospitalization event for a subject patient may be determined. The patient score may be based on the trained ensemble classifier being applied to a patient vector for the subject patient. The patient score may be determined by generating one or more dependent patient scores, each indicative of a respective likelihood of the hospitalization event for the subject patient. The one or more dependent patient scores may be based on the one or more classifiers applied to the subject vector for the subject patient. The patient score may be determined based on a meta-classifier applied to the one or more dependent patient scores. The meta-classifier may include a logistic regression algorithm. The patient score may be provided to a second computing device, such as a reporting subsystem.

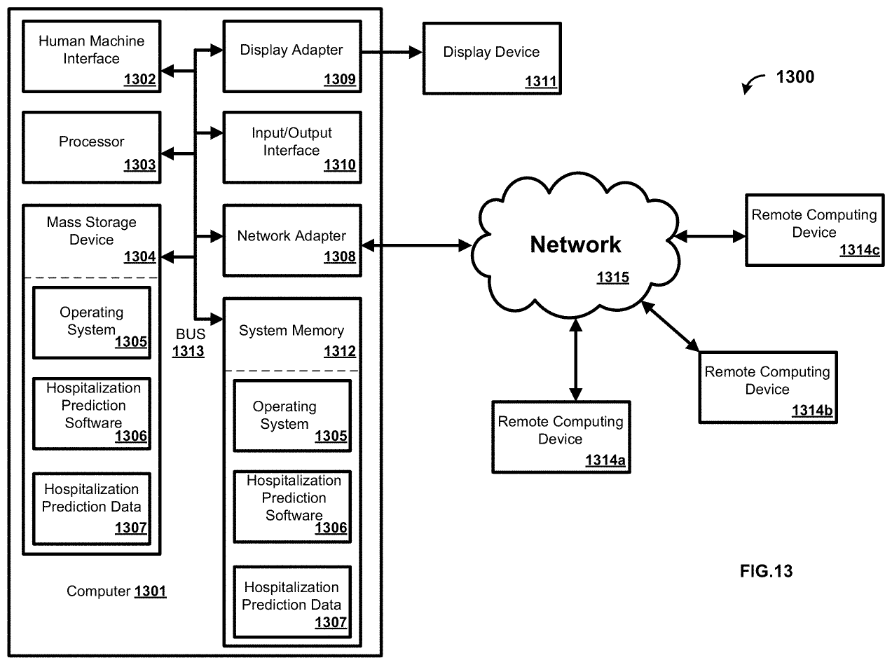

FIG. 13 shows a block diagram of an example computing device 1300 for improved predictive analytics, such as patient scoring and hospitalization prediction, in accordance with the present description. Any of the devices/subsystems shown in FIGS. 2 and 9 may each be a computer 1301 as shown in FIG. 13. The computer 1301 may include one or more processors 1303, a system memory 1312, and a bus 1313 that couples various system components including the one or more processors 1303 to the system memory 1312. In the case of multiple processors 1303, the computer 1301 may utilize parallel computing. The bus 1313 is one or more of several possible types of bus structures, including a memory bus or memory controller, a peripheral bus, an accelerated graphics port, or local bus using any of a variety of bus architectures.

The computer 1301 may operate on and/or comprise a variety of computer readable media (e.g., non-transitory media). The readable media may be any available media that is accessible by the computer 1301 and may include both volatile and non-volatile media, removable and non-removable media. The system memory 1312 has computer readable media in the form of volatile memory, such as random access memory (RAM), and/or non-volatile memory, such as read only memory (ROM). The system memory 1312 may store data such as hospitalization prediction data 1307 and/or program modules such as the operating system 1305 and hospitalization prediction software 1306 that are accessible to and/or are operated on by the one or more processors 1303. The hospitalization prediction software 1306 may use the hospitalization prediction data 1307 to perform content scoring using the VVQS formula and methods described above. For example, one or more output metrics associated with output of a content segment at a user device may be determined by the computer 1301 using the hospitalization prediction software 1306 and the hospitalization prediction data 1307. The one or more output metrics may be stored in the system memory 1312.

The computer 1301 may also have other removable/non-removable, volatile/non-volatile computer storage media. FIG. 13 shows the mass storage device 1304 which may provide non-volatile storage of computer code, computer readable instructions, data structures, program modules, and other data for the computer 1301. The mass storage device 1304 may be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Any number of program modules may be stored on the mass storage device 1304, such as the operating system 1305 and the hospitalization prediction software 1306. Each of the operating system 1305 and the hospitalization prediction software 1306 (e.g., or some combination thereof) may have elements of the program modules and the hospitalization prediction software 1306. The hospitalization prediction data 1307 may also be stored on the mass storage device 1304. The hospitalization prediction data 1307 may be stored in any of one or more databases known in the art. Such databases may be DB2®, Microsoft® Access, Microsoft® SQL Server, Oracle®, mySQL, PostgreSQL, and the like. The databases may be centralized or distributed across locations within the network 1315.

A user may enter commands and information into the computer 1301 via an input device (not shown). Examples of such input devices comprise, but are not limited to, a keyboard, pointing device (e.g., a computer mouse, remote control), a microphone, a joystick, a scanner, tactile input devices such as gloves, and other body coverings, motion sensor, and the like These and other input devices may be connected to the one or more processors 1303 via a human machine interface 1302 that is coupled to the bus 1313, but may be connected by other interface and bus structures, such as a parallel port, game port, an IEEE 1394 Port (also known as a Firewire port), a serial port, network adapter 1308, and/or a universal serial bus (USB).

The display device 1311 may also be connected to the bus 1313 via an interface, such as the display adapter 1309. It is contemplated that the computer 1301 may have more than one display adapter 1309 and the computer 1301 may have more than one display device 1311. The display device 1311 may be a monitor, an LCD (Liquid Crystal Display), light emitting diode (LED) display, television, smart lens, smart glass, and/or a projector. In addition to the display device 1311, other output peripheral devices may be components such as speakers (not shown) and a printer (not shown) which may be connected to the computer 1301 via the Input/Output Interface 1310. Any step and/or result of the methods may be output (or caused to be output) in any form to an output device. Such output may be any form of visual representation, including, but not limited to, textual, graphical, animation, audio, tactile, and the like. The display device 1311 and computer 1301 may be part of one device, or separate devices.

The computer 1301 may operate in a networked environment using logical connections to one or more remote computing devices 1314a,b,c. A remote computing device may be a personal computer, computing station (e.g., workstation), portable computer (e.g., laptop, mobile phone, tablet device), smart device (e.g., smartphone, smart watch, activity tracker, smart apparel, smart accessory), security and/or monitoring device, a server, a router, a network computer, a peer device, edge device, and so on. Logical connections between the computer 1301 and a remote computing device 1314a,b,c may be made via a network 1315, such as a local area network (LAN) and/or a general wide area network (WAN). Such network connections may be through the network adapter 1308. The network adapter 1308 may be implemented in both wired and wireless environments. Such networking environments are conventional and commonplace in dwellings, offices, enterprise-wide computer networks, intranets, and the Internet.

Application programs and other executable program components such as the operating system 1305 are shown herein as discrete blocks, although it is recognized that such programs and components reside at various times in different storage components of the computing device 1301, and are executed by the one or more processors 1303 of the computer. An implementation of the hospitalization prediction software 1306 may be stored on or sent across some form of computer readable media. Any of the described methods may be performed by processor-executable instructions embodied on computer readable media.

While specific configurations have been described, it is not intended that the scope be limited to the particular configurations set forth, as the configurations herein are intended in all respects to be possible configurations rather than restrictive. Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; the number or type of configurations described in the specification.

It will be apparent to those skilled in the art that various modifications and variations may be made without departing from the scope or spirit. Other configurations will be apparent to those skilled in the art from consideration of the specification and practice described herein. It is intended that the specification and described configurations be considered as exemplary only, with a true scope and spirit being indicated by the following claims.

1. A method comprising:
receiving, by a computing device, a plurality of data records associated with a plurality of patients;
generating, based on the plurality of data records, a training dataset comprising a plurality of vectors each corresponding to a respective patient of the plurality of patients;
training, based on the training dataset, an ensemble classifier;
determining, based on the trained ensemble classifier, a patient score indicative of a likelihood of a hospitalization event for a subject patient; and
sending, by the computing device, the patient score to a reporting subsystem.

2. The method of claim 1, wherein each of the plurality of vectors comprises a health condition score based on the Charlson Comorbidity Index.

3. The method of claim 1, wherein each of the plurality of vectors comprises a Karnofsky Scale score, and wherein the method further comprises:
determining, based on the Karnofsky Scale score of each of the plurality of vectors, a performance status score ranging from 0 to 4.

4. The method of claim 1, wherein the ensemble classifier comprises one or more classifiers.

5. The method of claim 4, wherein the one or more classifiers comprises one or more of a random forest classifier, a naïve Bayes classifier, a gradient boosting machine classifier, an adaptive boosting classifier, or a logistic regression classifier.

6. The method of claim 5, wherein determining, based on the trained ensemble classifier, a patient score indicative of a likelihood of a hospitalization event for the subject patient comprises:
generating, based on the one or more classifiers applied to the subject vector for the subject patient, one or more dependent patient scores each indicative of a respective likelihood of the hospitalization event for the subject patient; and
determining, based on a meta-classifier and the one or more dependent patient scores, the patient score indicative of the likelihood of the hospitalization event for the subject patient, wherein the meta-classifier comprises a logistic regression algorithm.

7. The method of claim 4, wherein the one or more classifiers are selected for the ensemble based on one or more of an F-1 score, a precision, a recall, an accuracy, or a confusion metric for each of the one or more classifiers.

8. A method comprising:
generating, by a computing device based on a trained ensemble classifier, one or more dependent patient scores each indicative of a respective likelihood of a hospitalization event for a subject patient;
determining, based on a meta-classifier and the one or more dependent patient scores, a patient score indicative of the likelihood of the hospitalization event for the subject patient, wherein the meta-classifier comprises a logistic regression algorithm; and
sending, by the computing device, the patient score to a reporting subsystem.

9. The method of claim 8, further comprising:
receiving, by the computing device, a plurality of data records associated with a plurality of patients;
generating, based on the plurality of data records, a training dataset comprising a plurality of vectors each corresponding to a respective patient of the plurality of patients; and
training an ensemble classifier using the training dataset.

10. The method of claim 9, wherein each of the plurality of vectors comprises a health condition score based on the Charlson Comorbidity Index.

11. The method of claim 9, wherein each of the plurality of vectors comprises a Karnofsky Scale score, and the method further comprises:
determining, based on the Karnofsky Scale score of each of the plurality of vectors, a performance status score ranging from 0 to 4.

12. The method of claim 9, wherein the ensemble classifier comprises one or more classifiers.

13. The method of claim 12, wherein the one or more classifiers comprises one or more of a random forest classifier, a naïve Bayes classifier, a gradient boosting machine classifier, an adaptive boosting classifier, or a logistic regression classifier.

14. The method of claim 12, wherein the one or more classifiers are selected for the ensemble based on one or more of an F-1 score, a precision, a recall, an accuracy, or a confusion metric for each of the one or more classifiers.

15. A method comprising:
receiving, by a computing device, a plurality of data records associated with a plurality of patients;
generating, based on the plurality of data records, a training dataset comprising a plurality of vectors each corresponding to a respective patient of the plurality of patients; and
training an ensemble classifier using the training dataset.

16. The method of claim 15 further comprising:
generating, based on the trained ensemble classifier applied to the subject vector, one or more dependent patient scores associated with a subject vector for a subject patient, wherein each of the one or more dependent patient scores are indicative of a respective likelihood of the hospitalization event for the subject patient;
determining, based on a logistic regression algorithm and the one or more dependent patient scores, a patient score indicative of a likelihood of a hospitalization event for the subject patient; and
sending, by the computing device, the patient score to a reporting subsystem.

17. The method of claim 15, wherein each of the plurality of vectors comprises a standardized Karnofsky Scale score and a health condition score, and wherein each of the health condition scores are based on the Charlson Comorbidity Index.

18. The method of claim 17, further comprising:
determining, based on the standardized Karnofsky Scale score of each of the plurality of vectors, a performance status score ranging from 0 to 4.

19. The method of claim 15, wherein the ensemble classifier comprises one or more classifiers.

20. The method of claim 19, wherein the ensemble of the one or more classifiers comprises one or more of a random forest classifier, a naïve Bayes classifier, a gradient boosting machine classifier, an adaptive boosting classifier, or a logistic regression classifier.
